(19) United States
(12) Patent Application Publication
(10) Pub. No.: US 2007/0037135 A1
(43) Pub. Date: Feb. 15, 2007

(54) System and method for the identification and quantification of a biological sample suspended in a liquid

(76) Inventors: Russell H. Barnes, Columbus, OH (US); Gal Ingber, Oranat (IL); Jonathan Gurfinkel, Omer (IL)

Correspondence Address: THE WEBB LAW FIRM, P.C., 700 KOPPERS BUILDING, 436 SEVENTH AVENUE, PITTSBURGH, PA, 15219, US

(21) Appl. No.: 11/500,163

(22) Filed: Aug. 7, 2006

Related U.S. Application Data
(60) Provisional application No. 60/706,489, filed on Aug. 8, 2005.

(51) Int. Cl. C12Q 1/00; G06F 19/00; C12M 1/34; G01J 3/42

(52) U.S. Cl. 435/4; 435/287.2; 702/19; 356/319

(57) ABSTRACT

A system for the identification and quantification of a biological sample suspended in a liquid includes a fluorescence excitation module with at least one excitation light source; a sample interface module optically coupled to the fluorescence excitation module for positioning a biological sample to receive excitation light from the at least one excitation light source; a fluorescence emission module optically coupled to the sample interface module and comprising at least one detection device for detecting fluorescence excitation-emission matrices of the biological sample; and a computer module operatively coupled to the fluorescence emission module. The computer module performs multivariate analysis on the fluorescence excitation-emission matrices of the biological sample to identify and quantify the biological sample. The multivariate analysis may comprise extended partial least squared analysis for identification and quantification of the biological sample. A method for the identification and quantification of a biological sample suspended in a liquid is also provided.

### CROSS REFERENCE TO RELATED APPLICATIONS

This application claims the benefit of U.S. Provisional Patent Application No. 60/706,489 entitled “System for the Identification and Quantification of Biological Sample Suspended in Liquids” filed Aug. 8, 2005, which is hereby incorporated by reference in its entirety.

### BACKGROUND OF THE INVENTION

1. Field of the Invention

The present invention relates, in general, to a system and method for the identification and quantification of a biological sample suspended in a liquid. More specifically, the present invention relates to a system and method that utilizes multivariate analysis on fluorescence excitation-emission matrices of the biological sample to identify and quantify the biological sample.

2. Description of Related Art

In bacteriology, staining methods are used to identify two general groups of bacteria, Gram positive and Gram negative, without identifying the species. Chromogenic media may be used to isolate and identify some of the microorganisms involved in human pathology, but it cannot identify all the possible species. Currently it is possible to identify around 20,000 different bacterial species utilizes chemical staining methods. However, the great difficulty that still exists with such methods is the time of bacterial identification, which, for standard chemical methods using automated equipments, is between 18 and 24 hours having an isolated organism (which takes approximately an additional 24 hours).

In order to achieve faster response times, various spectrometric techniques have been developed. For instance, Fourier Transform Infrared (FTIR) spectroscopy has been utilized to achieve faster response times during analysis of biological samples. The steps required for FTIR spectroscopy are as follows. First, a group of bacteria isolated from the urine of patients with urinary tract infection (UTI) were collected. Prior to analysis the samples were oven-dried at 50° C. for 30 minutes. The spectra of these samples were collected over the 4000 cm⁻¹ to 600 cm⁻¹ wave-number range. The spectra were acquired at a rate of 20 Hz. To improve the signal-to-noise ratio, 256 spectra were co-added and averaged. The analysis of the information showed that identification of the samples could be performed.

Raman spectroscopy was also investigated as a possible method for identifying and quantifying a biological sample. The steps required for Raman spectroscopy are as follows. First, spectra were collected using a dispersive Raman spectrometer (Ramascope) with a low power (30 mW) near-infrared 780 nm diode laser with the power at the sampling point typically at 3 mW. Samples were presented as bacterial suspensions (3×10⁹ cells per ml). The spectrum was collected for 60 s. The analysis of the information showed that identification of the biological sample could be performed. However, the identification was not performed with high confidence.

Additional studies have proposed the use of fluorescence spectra for rapid bacterial identification. For instance, a method has been proposed that uses multi-excitation fluorescence spectroscopy which allows for the selection of the best excitation wavelength and consequently the selective excitation of biological molecular groups, for best bacteria species identification.

U.S. Pat. No. 6,834,237 to Noergaard et al. discloses a method of tracing a classification system for characterizing an isolated biological sample with respect to at least one condition comprising an isolated biological sample for an animal. The isolated biological sample is selected from body fluids or from a tissue sample. The tissue sample is not associated with the condition or conditions. An example of such a method would be taking urine samples from smokers and non-smokers and seeing if emitted light from a urine sample can detect if the person smokes.

U.S. Pat. Nos. 6,773,922, 6,426,045, 6,365,109 and 6,087,182, each to Jeng et al., disclose an apparatus and method for determining a parameter, such as the concentration of at least one analyte of a biological sample. The apparatus and method obtains such concentration values by using visible light absorption spectroscopy for certain analytes or infrared light absorption spectroscopy for other analytes.

U.S. Pat. No. 5,938,617 to Vo-Dinh is directed to a system which identifies biological pathogens in a sample by exciting a sample with light at several wavelengths and synchronously sampling the emission intensities. The system includes mechanisms for exposing the sample to excitation radiation and thereby generating an emission radiation. The biological pathogens may be viruses and bacteria.

However, each of the methods and/or systems discussed above involve either the use of reagents or requires sophisticated operator sample preparation that make the methods and/or systems more difficult to operate and more prone to operator mistakes. The time which is needed for such sample preparation also makes the methods and/or systems discussed above unsuitable for rapid diagnostics.

### SUMMARY OF THE INVENTION

Accordingly, it is an object of the present invention to provide a system that identifies and quantifies a biological sample in a fluid. It is a further object of the present invention to provide such a system that performs the analysis in a rapid manner. Another object of the present invention is to provide a system for identifying and quantifying a biological sample in a fluid that does not require the use of reagents in order to reduce the cost of material.

The present invention is directed to a system for the identification and quantification of a biological sample suspended in a liquid. The system includes a fluorescence excitation module with at least one excitation light source; a sample interface module optically coupled to the fluorescence excitation module for positioning a biological sample to receive excitation light from the at least one excitation light source; a fluorescence emission module optically coupled to the sample interface module and comprising at least one detection device for detecting fluorescence excitation-emission matrices of the biological sample; and a computer module operatively coupled to the fluorescence emission module. The computer module performs multivariate analysis on the fluorescence excitation-emission matrices of the biological sample to identify and quantify the biological sample. The multivariate analysis may comprise extended partial least squared analysis for identification and quantification of the biological sample.

The system may further include an absorption module and a diffuse-reflectance module. The absorption module uses light from either the at least one excitation light source or a separate modulated light source to perform absorption measurements on the biological sample. The absorption measurements may be combined with the fluorescence excitation-emission matrices of the biological sample to identify and quantify the biological sample. The absorption module may be either a monochromator or a filter wheel with a photomultiplier tube. The diffuse-reflectance module uses light from either the at least one excitation light source or a separate modulated light source to perform diffuse-reflectance measurements on the biological sample. The diffuse-reflectance measurements may be combined with the fluorescence excitation-emission matrices of the biological sample to identify and quantify the biological sample. The diffuse-reflectance module may be a monochromator, with a diode detector or a photomultiplier tube.

The at least one excitation light source may be a continuous light source, a pulsed flashlamp, a diode laser, a tunable laser or any combination thereof. The wavelength of the at least one excitation light source may be selectable through the use of grating monochromators, filter wheels with narrow bandpass filters, acousto-optic tunable filters, liquid crystal tunable filters, circular variable filters, linear variable filters or any combination thereof.

The at least one detection device of the fluorescence emission module may be either a scanning grating monochromator with a solid-state detector or a nonscanning grating monochromator with a multichannel array detector. The fluorescence emission module may further comprise gated electronics that control the depth of optical sampling in the liquid and optimize signal-to-noise characteristics.

The system may further comprise a display device for displaying the identification and quantification of the biological sample.

The present invention is further directed to a method of identifying and quantifying a biological sample suspended in a liquid. The method includes the steps of: a) providing a source of excitation light; b) exciting the biological sample with the source of excitation light; c) detecting spectral information from the biological sample in the form of excitation-emission matrices, absorption measurements, diffuse-reflectance measurements or any combination thereof; and d) performing multivariate analysis on the spectral information to identify and quantify the biological sample. The multivariate analysis may comprise extended partial least squared analysis for identification and quantification of the biological sample.

The source of excitation light may be a continuous light source, a pulsed flashlamp, a diode laser, a tunable laser or any combination thereof. The wavelength of the source of excitation light may be selectable through the use of grating monochromators, filter wheels with narrow bandpass filters, acousto-optic tunable filters, liquid crystal tunable filters, circular variable filters, linear variable filters or any combination thereof.

The method may further comprise the step of: e) displaying the identification and quantification of the biological sample. Data formatting and data pre-processing may be performed prior to step d). The multivariate analysis may comprise extended partial least squared analysis for identification and quantification of the biological sample.

These and other features and characteristics of the present invention, as well as the methods of operation and functions of the related elements of structures, will become more apparent upon consideration of the following description and the appended claims with reference to the accompanying drawings, all of which form a part of this specification, wherein like reference numerals designate corresponding parts in the various figures. As used in the specification and the claims, the singular form of “a”, “an”, and “the” include plural referents unless the context clearly dictates otherwise.

### BRIEF DESCRIPTION OF THE DRAWINGS

FIG. 1 is a general schematic view of a system for the identification and quantification of a biological sample suspended in a liquid in accordance with the present invention;

FIG. 2 is a schematic view of a right-angle configuration of a system for the identification and quantification of a biological sample suspended in a liquid in accordance with the present invention;

FIG. 3 is a schematic view of a front-face configuration of a system for the identification and quantification of a biological sample suspended in a liquid in accordance with the present invention;

FIG. 4 is a detailed schematic view of a sample interface module of the front-face configuration illustrated in FIG. 3;

FIG. 5 is a graph illustrating subtracted front-face fluorescence intensities as a function of Klebsiella pneumoniae concentration in a phosphate buffer solution;

FIG. 6 is a graph illustrating the subtracted fluorescence emission intensity as a function of the excitation wavelength for Klebsiella pneumoniae in a phosphate buffer solution;

FIG. 7 is a graph illustrating subtracted right-angle fluorescence intensities as a function of E. Coli concentration in water;

FIG. 8 is a graph illustrating subtracted front-face fluorescence intensities as a function of E. Coli concentration in a phosphate buffer solution; and

FIG. 9 is a graph illustrating subtracted right-angle fluorescence intensities as a function of E. Coli concentration in human urine.

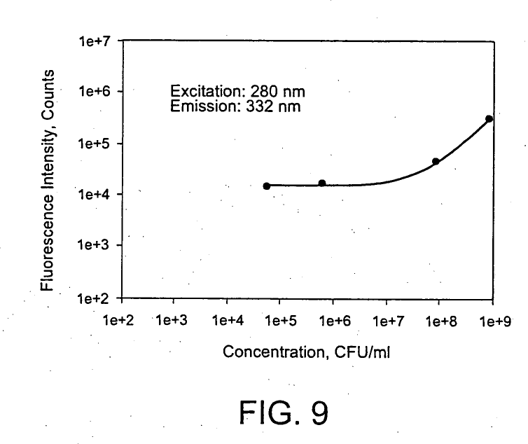

### DETAILED DESCRIPTION OF THE PRESENT INVENTION

For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The system and method of the present invention allows for the identification and quantification of a biological sample in a liquid in a rapid manner and without the addition of reagents to the liquid. The present invention is desirably used in such environments as point-of-care biomedical analyses of bacteria and viruses in human body fluids, identification of microorganisms in seawater ballasts for control of shipping entering U.S. coastal waters, detection and identification of biowarfare agents, the food and beverage industry and drinking and waste water contamination monitoring.

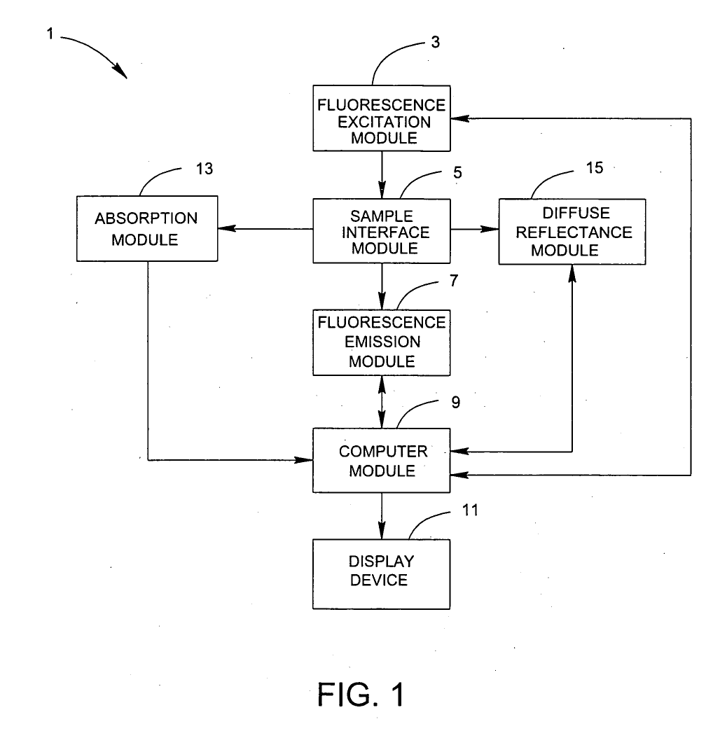

With reference to FIG. 1, a system, denoted generally by reference numeral 1, for the identification and quantification of a biological sample in a liquid comprises a fluorescence excitation module 3, a sample interface module 5, a fluorescence emission module 7, a computer module 9, a display device 11, an absorption module 13 and a diffuse-reflectance module 15.

Fluorescence excitation module 3, sample interface module 5, fluorescence emission module 7, absorption module 13 and a diffuse-reflectance module 15 are optically coupled to each other. Computer module 9 is operatively coupled to fluorescence excitation module 3, fluorescence emission module 7, absorption module 13 and a diffuse-reflectance module 15 and display device 11.

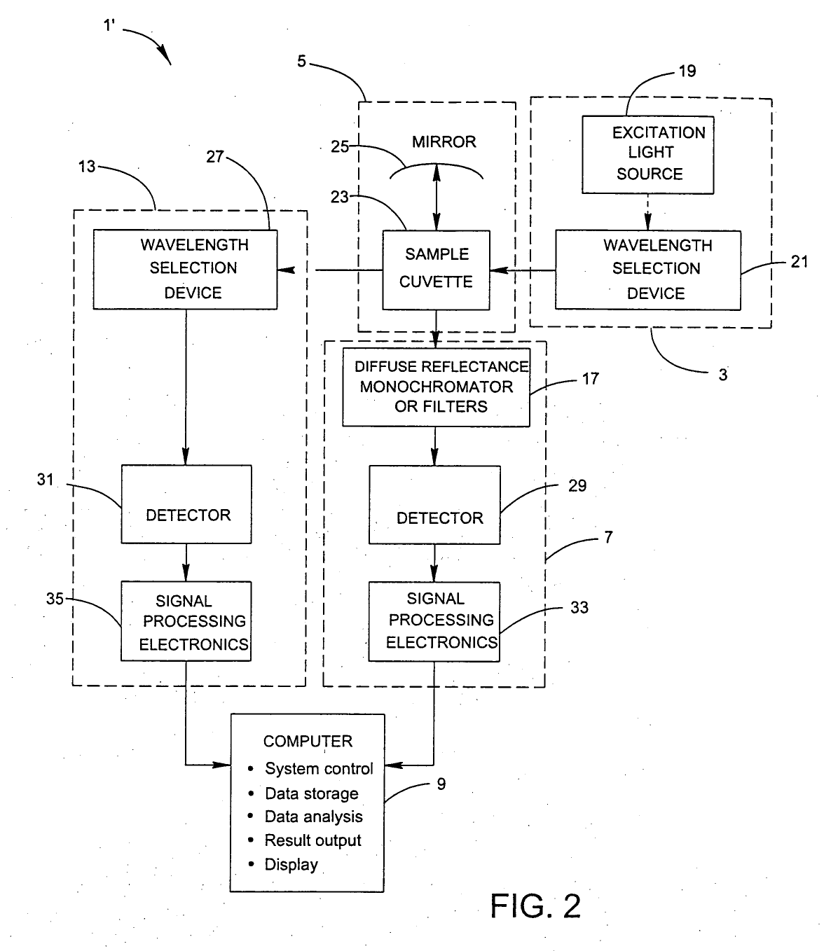

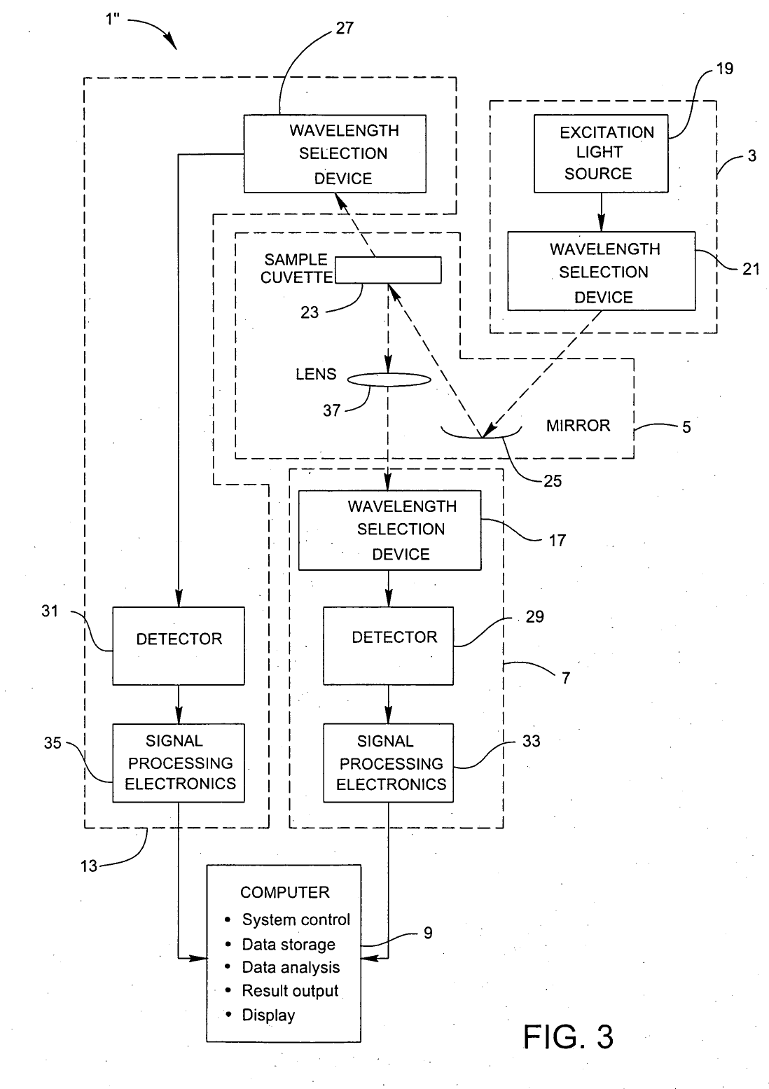

With reference to FIGS. 2 and 3, and with continuing reference to FIG. 1, system 1 of the present invention can be configured with a variety of optical arrangements for the excitation and collection of fluorescence. For instance, system 1 may be configured to have a right-angle configuration 1′ (see FIG. 2) or a front-face configuration 1″ (see FIG. 3).

Fluorescence excitation module 3 includes at least one excitation light source 19 and a wavelength selection device 21. Excitation light source 19 may be any suitable light source such as, but not limited to, a continuous light source such as a rare gas arc lamp or a deuterium lamp, a pulsed flashlamp, a diode laser or a tunable laser. Wavelength selection device 21 allows a user to select a specific wavelength for the light emitting from excitation light source 19. Wavelength selection device 21 may be any suitable device for selecting a wavelength from a light source including, but not limited to, grating monochromators, filter wheels with narrow bandpass filters, acousto-optic tunable filters (AOTFs), liquid crystal tunable filters (LCTFs), circular variable filters or linear variable filters.

Sample interface module 5 includes optical interfaces between fluorescence excitation module 3 and the biological sample in sample cuvettes 23 and polarization optics (not shown). Such sample cuvettes are well known in the art and are typically square or rectangular in shape (having a well area to contain the sample) and are made of a transparent material such as glass or a polymeric material.

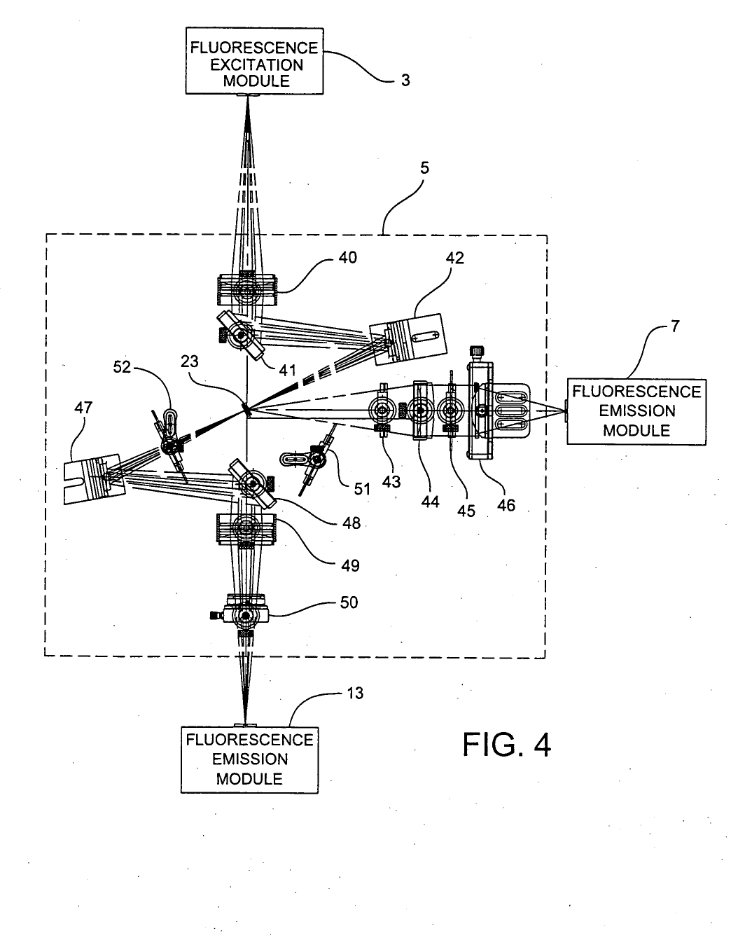

The optical interfaces include mirrors 25 and lens 37 and are provided to direct and focus the light produced by excitation light source 19 as appropriate. In an alternative embodiment of the present invention, single or bifurcated fiber optics may be provided as the optical interface. Sample cuvette 23 is provided to hold a biological sample suspended in a liquid in the appropriate position in the system. With reference to FIG. 4, a more detailed schematic diagram of sample interface module 5 of front-face configuration 1″ is provided. Sample interface 5 includes a lens 40 which focuses light provided by fluorescence emission module 3. Lens 40 may be a combination of a CVI PXF-50.8-90.8-UV lens and a CVI BXF-50.8-312.0-UV lens manufactured by CVI Laser LLC, 200 Dorado SE, Albuquerque, N. Mex. 87123. The light focused by lens 40 is then reflected by mirrors 41 and 42 to sample cuvette 23. Mirror 41 may be a Newport 20D10.AL2 and mirror 42 may be a Newport 10D10.AL2 manufactured by the Newport Corporation, 1791 Deere Avenue, Irvine, Calif. 92606.

The light reflected from sample cuvette 23 is directed through an aperture 43 and focused by lens 44, aperture 45 and lens 46 to fluorescence emission module 7. Lens 44 may be a CVI PXF-50.8-77.3-UV lens and lens 46 may be a CVI PXF-50.8-40.7-UV lens each manufactured by CVI Laser LLC.

The light that is transmitted through sample cuvette 23 is reflected by mirrors 47 and 48. Mirror 47 may be a Newport 10D10.AL2 and mirror 48 may be a Newport 20D10.AL2 manufactured by the Newport Corporation. The light is then focused by a lens 49 through an iris mechanism 50 and directed to absorption module 13. Lens 49 may be a combination of a CVI PXF-50.8-90.8-UV lens and a CVI BXF-50.8-312.0-UV lens manufactured by CVI Laser LLC.

All of the optical components of sample interface 5 are positioned through the use of appropriate holders such as the holders and positioning devices manufactured by Thorlabs, Inc., 435 Route 206 North, Newton, N.J. 07860. The optical interface may also include beam dumps 51 and 52 positioned as shown in FIG. 4 in order to reduce stray light from reflections inside the instrument.

Absorption module 13 uses light from either excitation light source 19 or a separate modulated light source (not shown) to perform absorption measurements on the biological sample in sample cuvette 23. Absorption module 13 may be, but is not limited to, either a monochromator or a filter wheel with a photomultiplier tube. Diffuse-reflectance module 15 also uses light from either excitation light source 19 or a separate modulated light source (not shown) to perform diffuse-reflectance measurements on the biological sample in sample cuvette 23. Diffuse-reflectance module 15 may be, but is not limited to, a monochromator, a diode detector or a photomultiplier tube.

The polarization optics (not shown) are desirably polarizers for excitation and/or emission beams and elastic scattering of light from the liquid medium suspending the biological sample.

Fluorescence emission module 7 includes a wavelength selection device 17, detector 29 and signal processing electronics 33. Detector 29 is optically coupled to wavelength selection device 17. Detector 29 and wavelength selection device 17 may be any suitable detection device including, but not limited to, a scanning grating monochromator with a solid-state detector or a nonscanning grating monochromator with a multichannel array detector. Fluorescence emission module 7 may further include a filter wheel with narrow-band filters (not shown) and a multimodal multiplex spectroscopy (MMS) monochromator (not shown). The MMS monochromator is optimized for extended area diffuse fluorescence sources. Signal processing electronics 33, which are well known in the art, are desirably gated electronics that control the depth of optical scanning in the liquid and optimize signal-to-noise characteristics.

Computer module 9 is provided for system operation and control. Computer module 9 formats and preprocesses data received from signal processing electronics 33, and also performs analysis on the data to determine the identification and quantification of the biological sample. By formatting the data from signal processing electronics 33, computer module 9 determines the fluorescence excitation-emission matrices and can also determine absorbance vs. wavelength over selected spectral regions and diffuse-reflectance vs. wavelength over selected spectral regions. Computer module 9 then preprocesses this information by mean centering and variance scaling, smoothing and differentiation, optimum filtering, absorption and scattering corrections to produce unperturbed fluorescence spectra and integration of fluorescence, absorption and diffuse-reflectance spectra in preparation for multivariate analysis. Finally, computer module 9 performs multivariate spectral analysis on the data to determine the identification and quantification of the biological sample. Such multivariate spectral analysis preferably includes extended partial least squared (e-PLS) analysis for classification and quantifying of the biological sample. Multiway chemometric procedures such as PARAFAC and the Tucker methods, artificial neural network (ANN) methods and support vector machine (SVM) methods may also be performed on the data.

Upon completion of the multivariate spectral analysis by computer module 9, display device 11 displays relevant information to the user. This information may include, but is not limited to, biological sample identification and quantification, identification probability and quantification statistics. Display device 11 may be any suitable display including, but not limited to, a CRT display, a plasma display, a rear-projection display, an LCD display or the like.

In operation, systems 1, 1′ and 1″ performs the following steps. First, excitation light is provided by excitation light source 19. The wavelength of the excitation light is selected using wavelength selection device 21 and is directed toward the biological sample in sample cuvette 23 by mirrors 25. The excitation light thereby excites the biological sample. Spectral information from the biological sample in the form of excitation-emission matrices, absorption measurements from absorption module 13 and diffuse-reflectance measurements from diffuse-reflectance module are detected by detector 29 and detector 31. This information is then processed by signal processing electronics 33 and 35. Next, data formatting and data pre-processing are performed on the spectral information by computer module 9. Computer module 9 then performs multivariate analysis comprising extended partial least squared analysis on the formatted and pre-processed spectral information to identify and quantify the biological sample. Finally, the identification and quantification of the biological sample is displayed for user interpretation.

Various embodiments of the invention will now be described by the following examples. The examples are intended to be illustrative only and are not intended to limit the scope of the invention.

### EXAMPLE 1

The following tables provide the excitation-emission matrices for a Phosphate Buffer Solution without (Table 1) and with (Table 2) Klebsiella pneumoniae at a concentration of 1.6×10⁷ CFU/mL. These excitation-emission matrices were produced using the front-face configuration of system 1 as illustrated in FIG. 3.

TABLE 1
(Without Klebsiella pneumoniae)
Fluorescence Intensity, Integrated Counts

| Emission | | | | | | |
|---|---|---|---|---|---|---|
| Wavelength, | Excitation Wavelength | | | | | |
| nm | 250 | 260 | 270 | 280 | 290 | 300 |
| 304 | 3.98E+02 | 3.19E+02 | 8.26E+02 | 5.01E+02 | 2.51E+02 | |
| 308 | 7.33E+02 | 7.46E+02 | 8.51E+02 | 3.30E+03 | 3.93E+02 | |
| 312 | 9.02E+02 | 7.85E+02 | 9.24E+02 | 7.13E+03 | 4.25E+02 | 1.38E+03 |
| 316 | 7.92E+02 | 8.25E+02 | 8.98E+02 | 2.73E+03 | 580E+02 | 1.31E+03 |
| 320 | 9.61E+02 | 8.21E+02 | 8.42E+02 | 8.96E+02 | 2.44E+03 | 1.26E+03 |
| 324 | 9.98E+02 | 8.87E+02 | 8.22E+02 | 7.64E+02 | 6.17E+03 | 1.23E+03 |
| 328 | 9.49E+02 | 9.69E+02 | 8.58E+02 | 8.31E+02 | 2.72E+03 | 1.49E+03 |
| 332 | 1.00E+03 | 9.47E+02 | 8.70E+02 | 8.66E+02 | 7.19E+02 | 3.65E+03 |
| 336 | 9.90E+02 | 8.04E+02 | 7.66E+02 | 8.31E+02 | 6.62E+02 | 5.95E+03 |
| 340 | 9.28E+02 | 7.60E+02 | 7.49E+02 | 7.12E+02 | 5.83E+02 | 3.67E+03 |
| 344 | 9.92E+02 | 7.38E+02 | 6.67E+02 | 6.27E+02 | 5.90E+02 | 1.39E+03 |
| 348 | 9.34E+02 | 6.87E+02 | 6.92E+02 | 6.88E+02 | 5.63E+02 | 1.28E+03 |
| 352 | 8.74E+02 | 7.79E+02 | 7.31E+02 | 6.63E+02 | 5.33E+02 | 1.67E+03 |
| 356 | 9.93E+02 | 8.43E+02 | 7.16E+02 | 6.38E+02 | 5.24E+02 | 1.64E+03 |
| 360 | 1.06E+03 | 9.49E+02 | 6.91E+02 | 6.54E+02 | 5.27E+02 | 1.04E+03 |
| 364 | 1.04E+03 | 8.17E+02 | 6.45E+02 | 5.93E+02 | 5.16E+02 | 1.17E+03 |
| 368 | 1.01E+03 | 8.47E+02 | 6.52E+02 | 5.36E+02 | 5.18E+02 | 1.19E+03 |
| 372 | 9.84E+02 | 7.88E+02 | 6.13E+02 | 5.31E+02 | 4.63E+02 | 1.50E+03 |
| 376 | 1.01E+03 | 7.65E+02 | 6.33E+02 | 5.12E+02 | 4.60E+02 | 2.45E+03 |
| 380 | 1.04E+03 | 7.83E+02 | 6.34E+02 | 5.15E+02 | 4.66E+02 | 1.64E+03 |
| 384 | 9.14E+02 | 7.41E+02 | 6.69E+02 | 5.15E+02 | 4.72E+02 | 1.43E+03 |
| 388 | 1.00E+03 | 7.29E+02 | 5.83E+02 | 5.06E+02 | 4.35E+02 | 1.28E+03 |
| 392 | 8.93E+02 | 8.30E+02 | 6.16E+02 | 4.98E+02 | 4.34E+02 | 1.46E+03 |
| 396 | 9.12E+02 | 8.17E+02 | 5.84E+02 | 5.16E+02 | 4.51E+02 | 1.93E+03 |
| 400 | 9.92E+02 | 7.56E+02 | 6.18E+02 | 4.88E+02 | 4.55E+02 | 1.22E+03 |
| 408 | 9.50E+02 | 7.83E+02 | 5.50E+02 | 4.53E+02 | 4.15E+02 | 1.17E+03 |
| 412 | 8.45E+02 | 7.57E+02 | 5.22E+02 | 4.95E+02 | 4.50E+02 | 1.17E+03 |
| 416 | 9.12E+02 | 7.53E+02 | 5.95E+02 | 4.74E+02 | 4.23E+02 | 1.62E+03 |
| 420 | 8.95E+02 | 8.54E+02 | 6.10E+02 | 4.83E+02 | 4.06E+02 | 1.47E+03 |
| 424 | 9.88E+02 | 8.98E+02 | 6.03E+02 | 4.77E+02 | 4.27E+02 | 1.62E+03 |
| 428 | 1.05E+03 | 8.44E+02 | 5.95E+02 | 4.81E+02 | 4.35E+02 | 1.49E+03 |
| 432 | 1.02E+03 | 8.50E+02 | 5.99E+02 | 4.55E+02 | 3.88E+02 | 1.38E+03 |
| 436 | 1.08E+03 | 9.45E+02 | 5.67E+02 | 4.72E+02 | 3.82E+02 | 1.62E+03 |
| 440 | 1.08E+03 | 8.54E+02 | 5.54E+02 | 4.72E+02 | 3.86E+02 | 1.50E+03 |

TABLE 2
(With Klebsiella pneumoniae)
Fluorescence Intensity, Integrated Counts

| Emission | | | | | | |
|---|---|---|---|---|---|---|
| Wavelength, | Excitation Wavelength | | | | | |
| nm | 250 | 260 | 270 | 280 | 290 | 300 |
| 304 | 4.12E+03 | 4.67E+03 | 6.29E+03 | 6.15E+03 | 3.03E+03 | |
| 308 | 9.23E+03 | 1.05E+04 | 1.39E+04 | 1.82E+04 | 8.28E+03 | |
| 312 | 1.05E+04 | 1.38E+04 | 1.88E+04 | 2.74E+04 | 1.23E+04 | 2.97E+03 |
| 316 | 1.19E+04 | 1.67E+04 | 2.34E+04 | 2.80E+04 | 1.63E+04 | 3.84E+03 |
| 320 | 1.29E+04 | 1.88E+04 | 2.65E+04 | 3.01E+04 | 2.16E+04 | 4.49E+03 |
| 324 | 1.33E+04 | 2.03E+04 | 2.88E+04 | 3.28E+04 | 2.70E+04 | 4.96E+03 |
| 328 | 1.31E+04 | 2.08E+04 | 3.00E+04 | 3.40E+04 | 2.50E+04 | 5.63E+03 |
| 332 | 1.28E+04 | 2.01E+04 | 3.02E+04 | 3.39E+04 | 2.31E+04 | 8.06E+03 |
| 336 | 1.20E+04 | 1.96E+04 | 2.86E+04 | 3.27E+04 | 2.25E+04 | 1.01E+04 |
| 340 | 1.10E+04 | 1.89E+04 | 2.73E+04 | 3.15E+04 | 2.19E+04 | 7.73E+03 |
| 344 | 1.05E+04 | 1.77E+04 | 2.57E+04 | 2.97E+04 | 2.03E+04 | 5.47E+03 |
| 348 | 9.87E+03 | 1.67E+04 | 2.42E+04 | 2.80E+04 | 1.91E+04 | 5.01E+03 |
| 352 | 9.42E+03 | 1.57E+04 | 2.36E+04 | 2.65E+04 | 1.86E+04 | 5.28E+03 |
| 356 | 9.02E+03 | 1.55E+04 | 2.19E+04 | 2.52E+04 | 1.78E+04 | 5.11E+03 |
| 360 | 8.57E+03 | 1.43E+04 | 2.08E+04 | 2.37E+04 | 1.68E+04 | 4.32E+03 |
| 364 | 7.88E+03 | 1.33E+04 | 1.94E+04 | 2.16E+04 | 1.53E+04 | 4.28E+03 |
| 368 | 6.87E+03 | 1.18E+04 | 1.66E+04 | 1.97E+04 | 1.38E+04 | 3.71E+03 |
| 372 | 6.45E+03 | 1.07E+04 | 1.51E+04 | 1.73E+04 | 1.21E+04 | 3.78E+03 |
| 376 | 5.71E+03 | 9.54E+03 | 1.40E+04 | 1.55E+04 | 1.10E+04 | 4.48E+03 |
| 380 | 5.13E+03 | 8.94E+03 | 1.21E+04 | 1.41E+04 | 9.95E+03 | 3.78E+03 |
| 384 | 5.00E+03 | 8.07E+03 | 1.10E+04 | 1.27E+04 | 8.88E+03 | 3.08E+03 |
| 388 | 4.62E+03 | 7.17E+03 | 1.00E+04 | 1.13E+04 | 7.89E+03 | 2.87E+03 |
| 392 | 3.94E+03 | 6.30E+03 | 8.71E+03 | 9.60E+03 | 7.00E+03 | 2.78E+03 |
| 396 | 3.17E+03 | 5.47E+03 | 7.61E+03 | 8.48E+03 | 6.07E+03 | 3.18E+03 |
| 400 | 3.31E+03 | 4.62E+03 | 6.56E+03 | 7.11E+03 | 4.91E+03 | 2.17E+03 |
| 404 | 2.72E+03 | 4.06E+03 | 5.37E+03 | 6.00E+03 | 4.30E+03 | 2.12E+03 |
| 408 | 2.38E+03 | 3.64E+03 | 4.78E+03 | 5.50E+03 | 3.73E+03 | 1.83E+03 |
| 412 | 2.30E+03 | 3.19E+03 | 4.11E+03 | 4.68E+03 | 3.33E+03 | 1.75E+03 |
| 416 | 2.33E+03 | 2.92E+03 | 3.97E+03 | 4.10E+03 | 2.91E+03 | 2.16E+03 |
| 420 | 2.05E+03 | 2.74E+03 | 3.49E+03 | 3.75E+03 | 2.74E+03 | 2.01E+03 |
| 424 | 2.09E+03 | 2.62E+03 | 3.25E+03 | 3.32E+03 | 2.46E+03 | 2.00E+03 |
| 428 | 1.88E+03 | 2.28E+03 | 2.82E+03 | 3.09E+03 | 2.17E+03 | 1.85E+03 |
| 432 | 1.92E+03 | 2.24E+03 | 2.44E+03 | 2.51E+03 | 1.86E+03 | 1.77E+03 |
| 436 | 1.91E+03 | 2.12E+03 | 2.31E+03 | 2.31E+03 | 1.62E+03 | 1.79E+03 |
| 440 | 1.79E+03 | 1.94E+03 | 2.04E+03 | 1.99E+03 | 1.46E+03 | 1.74E+03 |

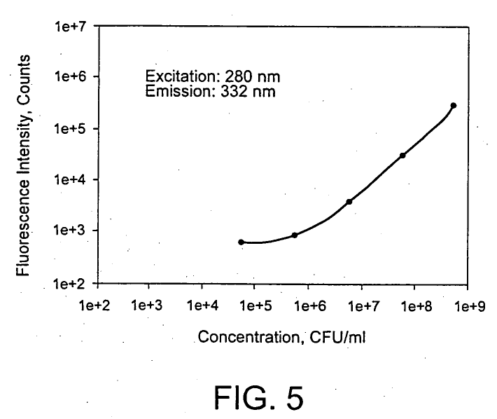

The above tables are summarized on the graphs of FIGS. 4 and 5. FIG. 4 illustrates front-face fluorescence intensities as a function of Klebsiella pneumoniae concentration in a phosphate buffer solution. FIG. 5 illustrates the subtracted fluorescence emission intensity as a function of the excitation wavelength for Klebsiella pneumoniae in a phosphate buffer solution.

### EXAMPLE 2

The following tables provide the excitation-emission matrices for water (Table 3) and water with E. Coli (Table 4) at a concentration of 3.9×10⁷ CFU/mL. These excitation-emission matrices were produced using the right-angle configuration of system 1 as in FIG. 2.

TABLE 3
(Water)
Fluorescence Intensity, Integrated Counts

| Emission | | | | | | |
|---|---|---|---|---|---|---|
| Wavelength, | Excitation Wavelength | | | | | |
| nm | 250 | 260 | 270 | 280 | 290 | 300 |
| 300 | 9.90E+02 | 8.81E+02 | 4.30E+03 | 5.44E+02 | | |
| 304 | 1.21E+03 | 1.04E+03 | 7.48E+02 | 1.71E+03 | 5.47E+02 | |
| 308 | 1.50E+03 | 1.21E+03 | 5.58E+02 | 6.75E+03 | 4.60E+02 | |
| 312 | 1.56E+03 | 1.27E+03 | 5.65E+02 | 6.12E+03 | 5.23E+02 | 5.80E+02 |
| 316 | 1.62E+03 | 1.35E+03 | 6.40E+02 | 1.44E+03 | 1.65E+03 | 5.72E+02 |
| 320 | 1.66E+03 | 1.39E+03 | 6.80E+02 | 8.85E+02 | 7.10E+03 | 5.29E+02 |
| 324 | 1.52E+03 | 1.26E+03 | 7.38E+02 | 8.93E+02 | 7.16E+03 | 5.03E+02 |
| 328 | 1.49E+03 | 1.24E+03 | 6.66E+02 | 8.55E+02 | 1.63E+03 | 1.54E+03 |
| 332 | 1.36E+03 | 1.22E+02 | 6.32E+02 | 7.27E+02 | 7.27E+02 | 7.46E+03 |
| 336 | 1.23E+03 | 9.97E+02 | 5.67E+02 | 6.88E+02 | 6.09E+02 | 7.92E+03 |
| 340 | 1.09E+03 | 8.90E+02 | 5.79E+02 | 6.05E+02 | 5.61E+02 | 1.78E+03 |
| 344 | 1.07E+02 | 8.68E+02 | 5.17E+02 | 5.31E+02 | 4.93E+02 | 5.87E+02 |
| 348 | 9.80E+02 | 7.50E+02 | 5.15E+02 | 7.03E+02 | 4.21E+02 | 5.05E+02 |
| 352 | 9.93E+02 | 7.78E+02 | 4.95E+02 | 6.02E+02 | 4.21E+02 | 3.98E+02 |
| 356 | 9.73E+02 | 8.06E+02 | 4.93E+02 | 5.37E+02 | 4.18E+02 | 3.22E+02 |
| 360 | 8.45E+02 | 7.36E+02 | 4.27E+02 | 4.93E+02 | 5.53E+02 | 2.78E+02 |
| 364 | 8.71E+02 | 7.60E+02 | 4.43E+02 | 5.82E+02 | 5.06E+02 | 3.37E+02 |
| 368 | 8.64E+02 | 7.19E+02 | 4.50E+02 | 5.40E+02 | 3.98E+02 | 3.32E+02 |
| 372 | 8.32E+02 | 6.66E+02 | 3.97E+02 | 4.92E+02 | 3.70E+02 | 4.82E+02 |
| 376 | 8.81E+02 | 6.61E+02 | 3.48E+02 | 4.48E+02 | 4.32E+02 | 5.11E+02 |
| 380 | 8.43E+02 | 7.02E+02 | 3.93E+02 | 4.60E+02 | 3.97E+02 | 3.53E+02 |
| 384 | 7.50E+02 | 6.79E+02 | 3.77E+02 | 4.63E+02 | 3.35E+02 | 3.82E+02 |
| 388 | 8.07E+02 | 6.37E+02 | 4.06E+02 | 4.74E+02 | 3.55E+02 | 4.07E+02 |
| 392 | 7.84E+02 | 5.51E+02 | 3.74E+02 | 4.82E+02 | 3.09E+02 | 4.64E+02 |
| 396 | 7.45E+02 | 6.02E+02 | 3.53E+02 | 4.28E+02 | 3.13E+02 | 3.56E+02 |
| 400 | 6.87E+02 | 5.70E+02 | 3.35E+02 | 4.58E+02 | 4.14E+02 | 3.50E+02 |

TABLE 4
(Water with E. Coli)
Fluorescence Intensity, Integrated Counts

| Emission | | | | | | |
|---|---|---|---|---|---|---|
| Wavelength, | Excitation Wavelength | | | | | |
| nm | 250 | 260 | 270 | 280 | 290 | 300 |
| 300 | 1.72E+03 | 2.01E+03 | 6.41E+03 | 3.73E+03 | | |
| 304 | 2.37E+03 | 2.95E+03 | 3.97E+03 | 6.56E+03 | 3.77E+03 | |
| 308 | 2.90E+03 | 3.72E+03 | 5.01E+03 | 1.37E+04 | 5.40E+03 | |
| 312 | 3.16E+03 | 4.39E+03 | 6.57E+03 | 1.45E+04 | 7.19E+03 | 2.42E+03 |
| 316 | 3.48E+03 | 5.10E+03 | 8.01E+03 | 1.22E+04 | 9.82E+03 | 2.87E+03 |
| 320 | 3.71E+03 | 5.51E+03 | 9.20E+03 | 1.31E+04 | 1.70E+04 | 3.25E+03 |
| 324 | 3.69E+03 | 5.91E+03 | 1.01E+04 | 1.45E+04 | 1.86E+04 | 3.58E+03 |
| 328 | 3.69E+03 | 5.95E+03 | 1.09E+04 | 1.52E+04 | 1.37E+04 | 5.10E+03 |
| 332 | 3.42E+03 | 6.07E+03 | 1.09E+04 | 1.52E+04 | 1.29E+04 | 1.14E+04 |
| 336 | 3.29E+03 | 5.72E+03 | 1.02E+04 | 1.45E+04 | 1.23E+04 | 1.19E+04 |
| 340 | 3.01E+03 | 5.30E+03 | 9.48E+03 | 1.34E+04 | 1.13E+04 | 5.33E+03 |
| 344 | 2.68E+03 | 4.96E+03 | 8.81E+03 | 1.28E+04 | 1.11E+04 | 3.74E+03 |
| 348 | 2.74E+03 | 4.77E+03 | 8.24E+03 | 1.24E+04 | 1.01E+04 | 3.63E+03 |
| 352 | 2.57E+03 | 4.62E+03 | 8.06E+03 | 1.14E+04 | 9.78E+03 | 3.55E+03 |
| 356 | 2.48E+03 | 4.27E+03 | 7.55E+03 | 1.05E+04 | 9.24E+03 | 3.32E+03 |
| 360 | 2.41E+03 | 4.03E+03 | 6.93E+03 | 9.87E+03 | 8.86E+03 | 3.13E+03 |
| 364 | 2.31E+03 | 3.78E+03 | 6.38E+03 | 8.98E+03 | 7.99E+03 | 2.76E+03 |
| 368 | 2.16E+03 | 3.50E+03 | 5.63E+03 | 7.91E+03 | 7.02E+03 | 2.61E+03 |
| 372 | 2.14E+03 | 3.21E+03 | 5.24E+03 | 7.37E+03 | 6.42E+03 | 2.76E+03 |
| 376 | 2.02E+03 | 3.13E+03 | 4.61E+03 | 6.57E+03 | 6.01E+03 | 2.81E+03 |
| 380 | 2.06E+03 | 2.73E+03 | 4.35E+03 | 5.86E+03 | 5.31E+03 | 2.19E+03 |
| 384 | 1.91E+03 | 2.55E+03 | 3.86E+03 | 5.31E+03 | 4.77E+03 | 2.10E+03 |
| 388 | 1.75E+03 | 2.41E+03 | 3.50E+03 | 5.01E+03 | 4.36E+03 | 1.96E+03 |
| 392 | 1.60E+03 | 2.08E+03 | 3.00E+03 | 4.35E+03 | 3.66E+03 | 1.99E+03 |
| 396 | 1.44E+03 | 1.99E+03 | 2.73E+03 | 3.77E+03 | 3.25E+03 | 1.54E+03 |
| 400 | 1.26E+03 | 1.64E+03 | 2.15E+03 | 3.40E+03 | 3.04E+03 | 1.42E+03 |

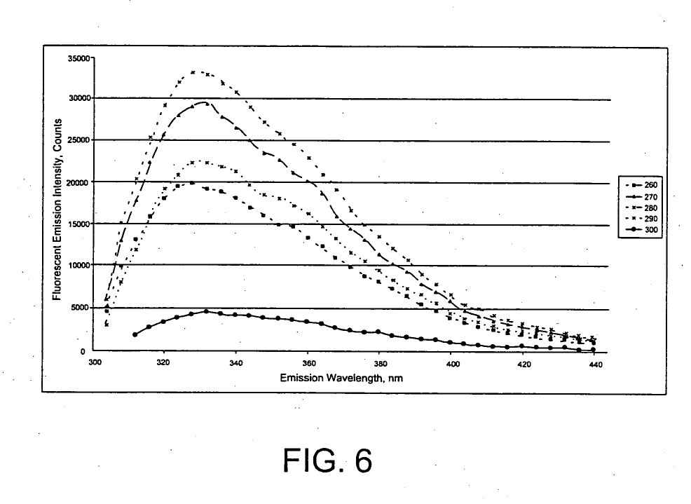

The above tables are summarized on the graph of FIG. 6. FIG. 6 illustrates subtracted right angle fluorescence intensities as a function of E. Coli concentration in water.

### EXAMPLE 3

The following tables provide the excitation-emission matrices for phosphate buffer solution without (Table 5) and with E. Coli (Table 6) at a concentration of 5.7×10⁷ CFU/mL. These excitation-emission matrices were produced using the front-face configuration of system 1 as illustrated in FIG. 3.

TABLE 5
(Without E. Coli)
Fluorescence Intensity, Integrated Counts

| Emission | | | | |
|---|---|---|---|---|
| Wavelength, | Excitation Wavelength | | | |
| nm | 270 | 280 | 290 | 300 |
| 304 | 9.31E+02 | 5.44E+02 | 2.75E+02 | |
| 308 | 8.68E+02 | 3.39E+03 | 4.00E+02 | |
| 312 | 8.75E+02 | 7.13E+03 | 4.13E+02 | 1.05E+03 |
| 316 | 8.68E+02 | 2.58E+03 | 5.40E+02 | 8.58E+02 |
| 320 | 8.46E+02 | 8.01E+02 | 2.40E+03 | 8.33E+02 |
| 324 | 7.79E+02 | 7.28E+02 | 6.10E+03 | 7.83E+02 |
| 328 | 7.89E+02 | 7.21E+02 | 2.60E+03 | 9.90E+02 |
| 332 | 7.61E+02 | 6.93E+02 | 6.06E+02 | 2.86E+03 |
| 336 | 6.90E+02 | 6.39E+02 | 5.25E+02 | 5.02E+03 |
| 340 | 6.40E+02 | 5.69E+02 | 4.89E+02 | 2.77E+03 |
| 344 | 5.89E+02 | 5.46E+02 | 4.52E+02 | 1.02E+03 |
| 348 | 5.65E+02 | 5.49E+02 | 4.56E+02 | 8.77E+02 |
| 352 | 5.82E+02 | 5.21E+02 | 4.37E+02 | 1.14E+03 |
| 356 | 5.56E+02 | 5.10E+02 | 4.35E+02 | 1.07E+03 |
| 360 | 5.80E+02 | 4.80E+02 | 4.31E+02 | 7.29E+02 |
| 364 | 5.54E+02 | 4.71E+02 | 4.35E+02 | 7.62E+02 |
| 368 | 5.29E+02 | 4.33E+02 | 3.99E+02 | 7.47E+02 |
| 372 | 5.03E+02 | 4.25E+02 | 3.81E+02 | 9.70E+02 |
| 376 | 4.75E+02 | 4.08E+02 | 3.55E+02 | 1.53E+03 |
| 380 | 4.96E+02 | 4.19E+02 | 3.65E+02 | 1.11E+03 |
| 384 | 4.80E+02 | 4.25E+02 | 3.67E+02 | 9.47E+02 |
| 388 | 4.85E+02 | 4.03E+02 | 3.61E+02 | 8.83E+02 |
| 392 | 4.71E+02 | 4.00E+02 | 3.51E+02 | 9.18E+02 |
| 396 | 4.56E+02 | 3.89E+02 | 3.57E+02 | 1.29E+03 |
| 400 | 4.47E+02 | 3.74E+02 | 3.46E+02 | 7.78E+02 |

TABLE 6
(With E. Coli)
Fluorescence Intensity, Integrated Counts

| Emission | | | | |
|---|---|---|---|---|
| Wavelength, | Excitation Wavelength | | | |
| nm | 270 | 280 | 290 | 300 |
| 304 | 2.62E+03 | 2.34E+03 | 1.13E+03 | |
| 308 | 4.60E+03 | 7.33E+03 | 2.42E+03 | |
| 312 | 6.04E+03 | 1.24E+04 | 3.51E+03 | 1.73E+03 |
| 316 | 7.10E+03 | 9.15E+03 | 4.50E+03 | 1.79E+03 |
| 320 | 7.91E+03 | 8.52E+03 | 7.31E+03 | 1.92E+03 |
| 324 | 8.62E+03 | 9.18E+03 | 1.14E+04 | 2.00E+03 |
| 328 | 9.11E+03 | 9.75E+03 | 8.50E+03 | 2.32E+03 |
| 332 | 9.30E+03 | 9.98E+03 | 6.78E+03 | 4.48E+03 |
| 336 | 9.03E+03 | 9.60E+03 | 6.48E+03 | 6.66E+03 |
| 340 | 8.44E+03 | 9.13E+03 | 6.15E+03 | 4.33E+03 |
| 344 | 8.02E+03 | 8.60E+03 | 5.89E+03 | 2.42E+03 |
| 348 | 7.73E+03 | 8.17E+03 | 5.70E+03 | 2.24E+03 |
| 352 | 7.50E+03 | 7.90E+03 | 5.40E+03 | 2.54E+03 |
| 356 | 7.16E+03 | 7.44E+03 | 5.20E+03 | 2.43E+03 |
| 360 | 6.73E+03 | 7.04E+03 | 4.80E+03 | 1.93E+03 |
| 364 | 6.23E+03 | 6.41E+03 | 4.46E+03 | 1.88E+03 |
| 368 | 5.59E+03 | 5.73E+03 | 3.97E+03 | 1.76E+03 |
| 372 | 5.06E+03 | 5.10E+03 | 3.57E+03 | 1.97E+03 |
| 376 | 4.56E+03 | 4.63E+03 | 3.24E+03 | 2.58E+03 |
| 380 | 4.22E+03 | 4.24E+03 | 2.94E+03 | 2.02E+03 |
| 384 | 3.83E+03 | 3.83E+03 | 2.62E+03 | 1.69E+03 |
| 388 | 3.43E+03 | 3.43E+03 | 2.40E+03 | 1.56E+03 |
| 392 | 3.02E+03 | 3.03E+03 | 2.13E+03 | 1.56E+03 |
| 396 | 2.65E+03 | 2.59E+03 | 1.87E+03 | 1.95E+03 |
| 400 | 2.22E+03 | 2.24E+03 | 1.57E+03 | 1.27E+03 |

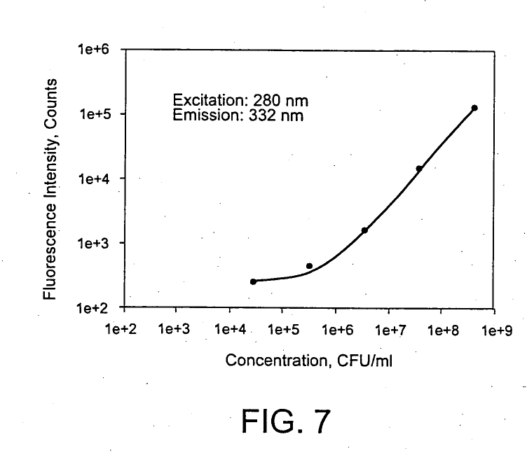

The above tables are summarized on the graph of FIG. 7. FIG. 7 illustrates subtracted front-face fluorescence intensities as a function of E. Coli concentration in a phosphate buffer solution.

### EXAMPLE 4

The following tables provide the excitation-emission matrices for Human Urine without (Table 7 ) and with E. Coli (Table 8) at a concentration of 8.9×10⁷ CFU/mL. These excitation-emission matrices were produced using the right-angle configuration of system 1 as illustrated in FIG. 2.

TABLE 7
(Human Urine)
Fluorescence Intensity, Integrated Counts

| Emission | | | | | |
|---|---|---|---|---|---|
| Wavelength, | Excitation Wavelength | | | | |
| nm | 260 | 270 | 280 | 290 | 300 |
| 300 | 5.12E+03 | 1.68E+04 | 2.21E+04 | | |
| 304 | 1.11E+04 | 3.51E+04 | 4.81E+04 | 1.81E+04 | |
| 308 | 2.27E+04 | 7.00E+04 | 9.75E+04 | 3.99E+04 | |
| 312 | 3.83E+04 | 1.14E+05 | 1.62E+05 | 7.75E+04 | 2.85E+04 |
| 316 | 5.53E+04 | 1.55E+05 | 2.24E+05 | 1.28E+05 | 5.77E+04 |
| 320 | 7.18E+04 | 1.93E+05 | 2.80E+05 | 1.87E+05 | 9.56E+04 |
| 324 | 8.95E+04 | 2.31E+05 | 3.38E+05 | 2.57E+05 | 1.43E+05 |
| 328 | 1.08E+05 | 2.72E+05 | 3.96E+05 | 3.32E+05 | 1.96E+05 |
| 332 | 1.26E+05 | 3.10E+05 | 4.51E+05 | 4.04E+05 | 2.47E+05 |
| 336 | 1.42E+05 | 3.46E+05 | 5.03E+05 | 4.71E+05 | 2.93E+05 |
| 340 | 1.58E+05 | 3.85E+05 | 5.58E+05 | 5.34E+05 | 3.34E+05 |
| 344 | 1.77E+05 | 4.33E+05 | 6.24E+05 | 6.11E+05 | 3.84E+05 |
| 348 | 2.04E+05 | 4.96E+05 | 7.19E+05 | 7.09E+05 | 4.42E+05 |
| 352 | 2.36E+05 | 5.78E+05 | 8.34E+05 | 8.26E+05 | 5.16E+05 |
| 356 | 2.69E+05 | 6.61E+05 | 9.54E+05 | 9.48E+05 | 5.94E+05 |
| 360 | 3.00E+05 | 7.35E+05 | 1.06E+06 | 1.06E+06 | 6.65E+05 |
| 364 | 3.25E+05 | 8.01E+05 | 1.16E+06 | 1.16E+06 | 7.34E+05 |
| 368 | 3.52E+05 | 8.65E+05 | 1.25E+06 | 1.25E+06 | 8.08E+05 |
| 372 | 3.77E+05 | 9.23E+05 | 1.34E+06 | 1.35E+06 | 8.88E+05 |
| 376 | 4.02E+05 | 9.81E+05 | 1.43E+06 | 1.44E+06 | 9.81E+05 |
| 380 | 4.25E+05 | 1.03E+06 | 1.51E+06 | 1.53E+06 | 1.09E+06 |
| 384 | 4.44E+05 | 1.07E+06 | 1.57E+06 | 1.61E+06 | 1.20E+06 |
| 388 | 4.55E+05 | 1.10E+06 | 1.61E+06 | 1.66E+06 | 1.31E+06 |
| 392 | 4.56E+05 | 1.09E+06 | 1.61E+06 | 1.68E+06 | 1.41E+06 |
| 396 | 4.51E+05 | 1.07E+06 | 1.58E+06 | 1.68E+06 | 1.49E+06 |
| 400 | 4.40E+05 | 1.04E+06 | 1.54E+06 | 1.65E+06 | 1.56E+06 |

TABLE 8
(Human Urine with E. Coli)
Fluorescence Intensity, Integrated Counts

| Emission | | | | | |
|---|---|---|---|---|---|
| Wavelength, | Excitation Wavelength | | | | |
| nm | 260 | 270 | 280 | 290 | 300 |
| 300 | 6.17E+03 | 1.98E+04 | 2.68E+04 | | |
| 304 | 1.31E+04 | 4.13E+04 | 5.70E+04 | 2.10E+04 | |
| 308 | 2.56E+04 | 8.08E+04 | 1.12E+05 | 4.57E+04 | |
| 312 | 4.32E+04 | 1.30E+05 | 1.83E+05 | 8.68E+04 | 3.15E+04 |
| 316 | 6.11E+04 | 1.74E+05 | 2.50E+05 | 1.42E+05 | 6.32E+04 |
| 320 | 7.88E+04 | 2.14E+05 | 3.12E+05 | 2.09E+05 | 1.06E+05 |
| 324 | 9.82E+04 | 2.56E+05 | 3.75E+05 | 2.85E+05 | 1.58E+05 |
| 328 | 1.20E+05 | 2.99E+05 | 4.39E+05 | 3.65E+05 | 2.16E+05 |
| 332 | 1.37E+05 | 3.41E+05 | 5.00E+05 | 4.47E+05 | 2.71E+05 |
| 336 | 1.55E+05 | 3.79E+05 | 5.55E+05 | 5.19E+05 | 3.18E+05 |
| 340 | 1.71E+05 | 4.19E+05 | 6.14E+05 | 5.87E+05 | 3.64E+05 |
| 344 | 1.94E+05 | 4.71E+05 | 6.86E+05 | 6.68E+05 | 4.14E+05 |
| 348 | 2.20E+05 | 5.37E+05 | 7.88E+05 | 7.71E+05 | 4.77E+05 |
| 352 | 2.54E+05 | 6.22E+05 | 9.10E+05 | 8.94E+05 | 5.53E+05 |
| 356 | 2.87E+05 | 7.07E+05 | 1.03E+06 | 1.03E+06 | 6.32E+05 |
| 360 | 3.20E+05 | 7.86E+05 | 1.15E+06 | 1.14E+06 | 7.07E+05 |
| 364 | 3.48E+05 | 8.54E+05 | 1.25E+06 | 1.24E+06 | 7.76E+05 |
| 368 | 3.74E+05 | 9.20E+05 | 1.35E+06 | 1.34E+06 | 8.53E+05 |
| 372 | 4.01E+05 | 9.82E+05 | 1.43E+06 | 1.44E+06 | 9.35E+05 |
| 376 | 4.24E+05 | 1.04E+06 | 1.53E+06 | 1.54E+06 | 1.03E+06 |
| 380 | 4.49E+05 | 1.10E+06 | 1.61E+06 | 1.63E+06 | 1.14E+06 |
| 384 | 4.67E+05 | 1.14E+06 | 1.68E+06 | 1.71E+06 | 1.26E+06 |
| 388 | 4.79E+05 | 1.16E+06 | 1.71E+06 | 1.77E+06 | 1.37E+06 |
| 392 | 4.82E+05 | 1.16E+06 | 1.71E+06 | 1.79E+06 | 1.48E+06 |
| 396 | 4.74E+05 | 1.13E+06 | 1.69E+06 | 1.78E+06 | 1.56E+06 |
| 400 | 4.63E+05 | 1.09E+06 | 1.64E+06 | 1.75E+06 | 1.63E+06 |

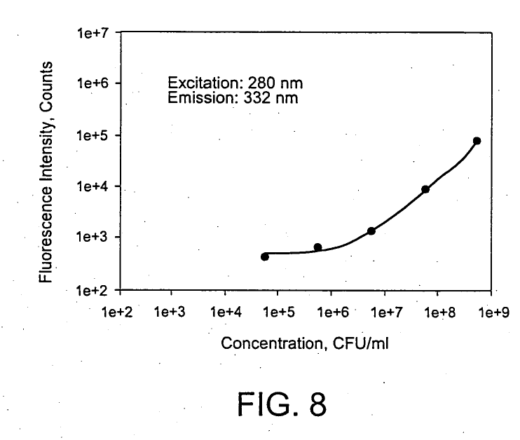

The above tables are summarized on the graph of FIG. 8. FIG. 8 illustrates subtracted right angle fluorescence intensities as a function of E. Coli concentration in human urine.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A system for the identification and quantification of a biological sample suspended in a liquid, the system comprising:
a fluorescence excitation module comprising at least one excitation light source;
a sample interface module optically coupled to the fluorescence excitation module for positioning a biological sample to receive excitation light from the at least one excitation light source and transmitting light to fluorescence, absorption and diffused reflectance modules;
a fluorescence emission module optically coupled to the sample interface module and comprising at least one detection device for detecting fluorescence excitation-emission matrices of the biological sample; and
a computer module operatively coupled to the fluorescence emission module,
wherein the computer module performs multivariate analysis on the fluorescence excitation-emission matrices of the biological sample to identify and quantify the biological sample.

2. The system of claim 1, wherein further comprises an absorption module and a diffuse-reflectance module.

3. The system of claim 2, wherein the absorption module uses light from one of the at least one excitation light source and a separate modulated light source to perform absorption measurements on the biological sample.

4. The system of claim 3, wherein the absorption measurements are combined with the fluorescence excitation-emission matrices of the biological sample to identify and quantify the biological sample.

5. The system of claim 2, wherein the absorption module is one of a monochromator or a filter wheel with a photomultiplier tube.

6. The system of claim 2, wherein the diffuse-reflectance module uses light from one of the at least one excitation light source and a separate modulated light source to perform diffuse-reflectance measurements on the biological sample.

7. The system of claim 6, wherein the diffuse-reflectance measurements are combined with the fluorescence excitation-emission matrices of the biological sample to identify and quantify the biological sample.

8. The system of claim 7, wherein the diffuse-reflectance module is one of a monochromator, a diode detector and a photomultiplier tube.

9. The system of claim 1, wherein the at least one excitation light source is a continuous light source, a pulsed flashlamp, a diode laser, a tunable laser or any combination thereof.

10. The system of claim 1, wherein a wavelength of the at least one excitation light source is selectable through the use of grating monochromators, filter wheels with narrow bandpass filters, acousto-optic tunable filters, liquid crystal tunable filters, circular variable filters, linear variable filters or any combination thereof.

11. The system of claim 1, wherein the at least one detection device of the fluorescence emission module is one of a scanning grating monochromator with a solid-state detector and a nonscanning grating monochromator with a multichannel array detector.

12. The system of claim 1, wherein the fluorescence emission module further comprises gated electronics that control the depth of optical sampling in the liquid and optimize signal-to-noise characteristics.

13. The system of claim 1, wherein the multivariate analysis comprises extended partial least squared analysis for identification and quantification of the biological sample.

14. The system of claim 1, further comprising a display device for displaying the identification and quantification of the biological sample.

15. A method of identifying and quantifying a biological sample suspended in a liquid, the method comprising the steps of:
a) providing a source of excitation light;
b) exciting the biological sample with the source of excitation light;
c) detecting spectral information from the biological sample in the form of excitation-emission matrices, absorption measurements, diffuse-reflectance measurements or any combination thereof; and
d) performing multivariate analysis on the spectral information to identify and quantify the biological sample.

16. The method of claim 15, wherein the source of excitation light is a continuous light source, a pulsed flashlamp, a diode laser, a tunable laser or any combination thereof.

17. The method of claim 15, wherein a wavelength of the source of excitation light is selectable through the use of grating monochromators, filter wheels with narrow bandpass filters, acousto-optic tunable filters, liquid crystal tunable filters, circular variable filters, linear variable filters or any combination thereof.

18. The method of claim 15, wherein the multivariate analysis comprises extended partial least squared for identification and quantification of the biological sample.

19. The method of claim 15, further comprising the step of:
e) displaying the identification and quantification of the biological sample.

20. The method of claim 15, wherein data formatting and data pre-processing are performed prior to step d).
